(19) United States
(12) Patent Application Publication
(10) Pub. No.: US 2006/0052448 A1
(43) Pub. Date: Mar. 9, 2006

(54) Use of methyl pyruvate or methyl pyruvic acid for the treatment of diseases of the nervous system and for protecting a human central nervous system against neuronal degeneration caused by defective intracellular energy production.

(75) Inventors: Stanley Charles Antosh, Palm Springs, CA (US); Anthony J. Meduri, New York City, NY (US)

Correspondence Address: OSMO, INC., 1271 VALDIVIA WAY, PALM SPRINGS, CA, 92262, US

(73) Assignees: Mr. Stanley Charles Antosh, Palm Springs, CA (US); Dr. Anthony J Meduri, New York City, NY (US)

(21) Appl. No.: 10/711,255

(22) Filed: Sep. 4, 2004

(51) Int. Cl. A61K 31/22; A61K 31/191

(52) U.S. Cl. 514/546; 514/557

(57) ABSTRACT

The present invention relates to the use of methyl pyruvic acid (a methyl ester of pyruvic acid) and/or methyl pyruvate (methyl pyruvate is the ionized form of methyl pyruvic acid) for the purpose of treating diseases of the nervous system and/or to prevent against neuronal degeneration due to defective intracellular energy production. Methyl pyruvate compounds can be used as therapeutically effective agents against a variety of diseases of the nervous system such as diabetic and toxic neuropathies, peripheral nervous system diseases, Alzheimer disease, Parkinson's disease, stroke, Huntington's disease, amyotropic lateral sclerosis, motor neuron disease, traumatic nerve injury, multiple sclerosis, dysmyelination, demyelination disorders, or cellular disorders which interfere with the energy metabolism of neurons and mitochondrial diseases. Use of methyl pyruvate and/or methyl pyruvic acid can be effective when administered orally or infused on either a chronic and/or acute basis. Treatment can be effective even when administered after the onset of an ischemic event that triggers neurodegeneration. In the following text, the terms “methyl pyruvate, methyl pyruvate compounds, methyl pyruvic acid” are used interchangeably.

### BACKGROUND OF INVENTION

Current U.S. Class: 514/23;514/565;514/275;514/385;514/386;514/396;514/557;514/501; 514/553; 514/563; 514/564; 514/575; 514/631; 514/636; 514/646; 514/546; 514/547

Intern'l Class:037/12; A61K 037/26; A61K 031/198,70,19,22

Field of Search:514/23, 3, 565, 275, 385, 386, 396, 546, 547, 553, 554, 501, 563, 564, 575, 631, 636, 646, 557

References Cited [Referenced By]

U.S. Patent Documents
U.S. Pat. No. 5,045,454 September, 1991 Bertheussen 435/29.
U.S. Pat. No. 5,091,404 February, 1992 Elgebaly 514/401.
U.S. Pat. No. 5,192,762 March, 1993 Gray et al. 514/249.
U.S. Pat. No. 5,210,098 May, 1993 Nath 514/577.
U.S. Pat. No. 5,321,030 June, 1994 Kaddurah-Daouk et al. 514/275.
U.S. Pat. No. 5,324,731 June, 1994 Kaddurah-Daouk et al. 514/275.
U.S. Pat. No. 5,741,661 April, 1998 Goldin et al. 435/29.

Foreign References:
EP0075805 1983-04 C07D 501/20 KYOWA HAKKO KOGYO CO., LTD Beta-lactam compound and a pharmaceutical composition containing the same.
EP0233780 1987-08 C07D 501/36 ELI LILLY AND COMPANYO-substituted oximino cephalosporins.
EP0370629 1990-05 C07C 251/60 IMPERIAL CHEMICAL INDUSTRIES PLC Fungicides EP0400805 1990-12 C07D 501/20 Ishimaru, Toshiyasu Cephalosporin compounds and their use.
EP0506149 1992-09 C07C 251/60 IMPERIAL CHEMICAL INDUSTRIES PLC Fungicides EP0581187 1994-02 C07C 251/54 ONO PHARMACEUTICAL CO., LTD. Oxime derivatives
EP0708098 1996-04 C07D 277/34 SANKYO COMPANY LIMITED Oxime derivatives, their preparation and their therapeutic use
EP0916651 1999-05 C07C 259/02 Sankyo Company, Limited PHENYLALKYLCARBOXYLIC ACID DERIVATIVES
JP59167576 1983-03, JP62077391 1984-12, JP9323929 1996-04,
JP11193272 1999-07 C07D 213/53 SANKYO CO LTD MEDICINE CONTAINING PHENYLALKYLCARBOXYLIC ACID DERIVATIVE
WO9602507 1996-02 C07D 215/14 ABBOTT LABORATORIES IMINOXYCARBOXYLATES AND DERIVATIVES AS INHIBITORS OF LEUKOTRIENE BIOSYNTHESIS WO9633724 1996-10 A61K 31/557 EVANS, Ronald, M. SELECTIVE MODULATORS OF PEROXISOME PROLIFERATOR ACTIVATED RECEPTOR-GAMMA, AND METHODS FOR THE USE THEREOF
WO9638427 1996-12 C07D 263/44 FUJIMOTO, Koichi AROMATIC OXYIMINO DERIVATIVES
WO9640128 1996-12 A61K 31/425 THE SALK INSTITUTE FOR BIOLOGICAL STUDIES MODULATORS OF PEROXISOME PROLIFERATOR ACTIVATED RECEPTOR-GAMMA, AND METHODS FOR THE USE THEREOF
WO9725042 1997-07 A61K 31/42 SMITH, STEPHEN, ALISTAIRUSE OF AN ANTAGONIST OF PPAR-ALPHA AND PPAR-GAMMA FOR THE TREATMENT OF SYNDROM X WO9731907 1997-09 C07D 263/56 BOSWELL, GRADY, EVANSUBSTITUTED 4-HYDROXY-PHENYLALCANOIC ACID DERIVATIVES WITH AGONIST ACTIVITY TO PPAR-GAMMA
WO9805331 1998-02 A61K 31/45 LIGAND PHARMACEUTICALS INCORPORATED OR TREATMENT OF TYPE 2 DIABETES OR CARDIOVASCULAR DISEASE WITH PPAR MODULATORS
WO9904815 1999-02 A61K 45/00 HASHIMOTO, SEIICHIMEDICINAL COMPOSITIONS WITH CHOLESTEROL-LOWERING EFFECT
WO9958510A1 issued Nov. 18, 1999 OXYIMINOALKANOIC ACID DERIVATIVES WITH HYPOGLYCEMIC AND HYPOLIPIDEMIC ACTIVITY

### FIELD OF THE INVENTION

The present invention relates to the field of neurology and relates to protecting the brain and central nervous system against damage due to neurological disorders or events in which energy-providing substrates (oxygen and glucose) are reduced or energy metabolism is suppressed or defective. More particularly to enhancing the production of energy by utilizing methyl pyruvic acid (a methyl ester of pyruvic acid) and/or methyl pyruvate (methyl pyruvate is the ionized form of methyl pyruvic acid), which modulate the system for the purpose of increasing neuronal energy production. In the following text, the terms “methyl pyruvate, methyl pyruvate compounds, methyl pyruvic acid” are used interchangeably.

It is an object of the present invention to provide treatment of diseases or events that affect cells of the nervous system by utilizing methyl pyruvate compounds, which modulate the system. The invention is in the field of neurology and relates to protecting the brain and central nervous system against damage due to neurological disorders or events in which energy-providing substrates (oxygen and glucose) are reduced or energy metabolism is suppressed or defective.

The nervous system is an un-resting assembly of cells that continually receives information, analyzes and perceives it and makes decisions. The principle cells of the nervous system are neurons and neuroglial cells. Neurons are the basic communicating units of the nervous system and possess dendrites, axons and synapses required for this role. Neuroglial cells consist of astrocytes, oligodendrocytes, ependymal cells, and microglial cells. Collectively, they are involved in the shelter and maintenance of neurons. The functions of astrocytes are incompletely understood but probably include the provision of biochemical and physical support and aid in insulation of the receptive surfaces of neurons. In addition to their activities in normal brain, they also react to CNS injury by glial scar formation. The principle function of the oligodendrocytes is the production and maintenance of CNS myelin. They contribute segments of myelin sheath to multiple axons. The ependyma cells react to injury mainly by cell loss. Microglial cells become activated and assume the shape of a macrophage in response to injury or destruction of the brain. These cells can also proliferate and adopt a rod-like form which could surround a tiny focus of necrosis or a dead neuron forming a glial nodule. Microglial degradation of dead neurons is called neuronophagia.

CNS neurons require energy to survive and perform their physiological functions, and it is generally recognized that the only source of energy for CNS neurons is the glucose and oxygen delivered by the blood. In recent years, neuroscientists have made considerable progress in understanding the mechanism by which energy deficiency leads to neuronal degeneration. There are two major components to the process by which cells utilize glucose and oxygen to produce energy. The first component entails anaerobic conversion of glucose to pyruvate, which releases a small amount of energy, and the second entails oxidative conversion of pyruvate to carbon dioxide and water with the release of a large amount of energy (these metabolic processes have been detailed in biochemical texts).

Pyruvate is continuously manufactured in the living organism, including the CNS, from glucose. The process by which glucose is converted to pyruvate involves a series of enzymatic reactions that occur anaerobically (in the absence of oxygen). This process is called “glycolysis”. A small amount of energy is generated in the glycolytic conversion of glucose to pyruvate, but a much larger amount of energy is generated in a subsequent more complicated series of reactions in which pyruvate is broken down to carbon dioxide and water. This process, which does require oxygen and is referred to as “oxidative respiration”, involves the stepwise metabolic breakdown of pyruvate by various enzymes of the Krebs tricarboxylic acid cycle and conversion of the products into high energy molecules by electron transport chain reactions.

It is recognized that various defects in the neuron's ability to utilize energy substates (glucose and oxygen) to maintain its energy levels can also trigger an excitotoxic process leading to death of neurons. It has been postulated that this is the mechanism by which neuronal degeneration occurs in neurological diseases such as Alzheimer's dementia, parkinsonism, Huntington's Chorea and amyotrophic lateral sclerosis. For example, evidence for defective intracellular energy metabolism has been found in samples of tissue removed by biopsy from the brains of patients with Alzheimer's disease and this has been proposed as the causative mechanism that triggers an unleashing of the excitotoxic potential of glutamate with death of neurons in Alzheimer's disease thereby being explained by an energy-linked excitotoxic process. Evidence for an intrinsic defect in intracellular energy metabolism has also been reported in parkinsonism and Huntington's Chorea.

If the blood supply to all or any portion of the CNS is shut off, thereby depriving neurons of both oxygen and glucose (a condition known as ischemia), the deprived neurons rapidly degenerate. This condition of inadequate blood flow is commonly known in clinical neurology as a “stroke.” If only the oxygen supply to the brain is interrupted, for example in asphyxia, suffocation or drowning, the condition is referred to as “hypoxia”. If only the glucose supply is disrupted, for example when a diabetic takes too much insulin, the condition is called “hypoglycemia”. All of these conditions involve energy deficiency and are recognized in clinical medicine as potential causes of brain damage. In recent years, neuroscientists have made considerable progress in understanding the mechanism by which energy deficiency leads to neuronal degeneration. Thus, rational therapeutic strategies for preventing neuronal degeneration in these disorders and events would include methods that correct energy deficiency.

Any pharmacologically acceptable salt can be used, provided that it is suitable and practical for administration to humans, sufficiently stable under reasonable storage conditions to have an adequate shelf life, and physiologically acceptable when introduced into the body by a suitable route of administration. The nature of the salt is not critical, provided that it is non-toxic and does not substantially interfere with the desired activity.

### OTHER REFERENCES

Rudinger, Peptide Hormones (June 1976: J. Parsons Ed.) p. 1-6.

Goodman & Gilman's, “The Pharmcological Basis of Therapeutics”, 6th Ed. (Macmillan Pub.: 1980) pp. 5-10. Chemical Abstracts AN 1982:49622, Bertolini et al., January 1981.

Chemical Abstracts AN 89:82648, Rees et al, U.S. Pat. No. 4,871,774, Mar. 10, 1989.* Annesley, T. and Walker, J., “Formation and Utilization of Novel High Energy Phosphate Reservoirs in Ehrlich Ascites Tumor Cells,” J. Biol. Chem., vol. 253, No. 22, 8120-5 (1978).

Beal, M., “Aging, Energy, and Oxidative Stress in Neurodegenerative Diseases,” Ann. Neurol., vol. 38, 357-66 (1995).

Beal, M., “Does Impairment of Energy Metabolism Result in Excitotoxic Neuronal Death in Neurodegenerative Illnesses?” Ann. Neurol., vol. 31, 119-30 (1992).

Beal, M. et al., “Age-Dependent Striatal Excitotoxic Lesions Produced by the Endogenous Mitochondrial Inhibitor Malonate,” J. Neurochem., vol. 61, 1147-50 (1993).

Brouillet, E. et al., “Chronic Mitochondrial Energy Impairment Produces Selective Striatal Degeneration and Abnormal Choreiform Movements in Primates,” PNAS, vol. 92, 7105-9 (1995).

Browne, S. et al., “Oxidative Damage and Metabolic Dysfunction in Huntington's Disease: Selective Vulnerability of the Basal Ganglia,” Ann. Neurol., vol. 41, 646-53 (1997).

Burbaeva, GSh et al., “Decreased Level of Immunoreactive Phosphokinase BB Isoenzymes in the Brain of Patients with Schizophrenia and Senile Dementia of the Alzheimer Type,” Zh. Nevropatol. Psikhiatr Im S S Korsakova, vol. 90, No. 7, 85-7 (1990)—abstract attached.

De Leon, M. et al., “Identification of Transcriptionally Regulated Genes After Sciatic Nerve Injury,” J. Neurosci. Res., vol. 29, 437-48 (1991).

Erecinska, M. and Silver, I., “ATP and Brain Function,” J. Cerebr. Bloof Flow and Metabolism, vol. 9, 2-19 (1989).

Gu, M. et al:, “Mitochondrial Defect in Huntington's Disease Caudate Nucleus,” Ann. Neurol., vol. 39, 385-9 (1996).

Gurney, M. et al., “Motor Neuron Degeneration in Mice That Express a Human Cu,Zn Superoxide Dismutase Mutation,” Science, vol. 264, 1772-5 (1994).

Henshaw, R. et al., “Malonate Produces Striatal Lesions by Indirect NMDA Receptor Activation,” Brain Research, vol. 647, 161-6 (1994).

Hertz, L. and Peng, L., “Energy Metabolism at the Cellular Level of the CNS,” Can. J. Physiol. Pharmacol., vol. 70, S145-57 (1992).

Ito, M., “The Cellular Basis of Cerebellar Plasticity,” Corr. Opin. Neurobiol., vol. 1, 616-20 (1991).

Jenkins, B. et al., “Evidence for Impairment of Energy Metabolism in Vivo in Huntington's Disease Using Localized .sup.1 H NMR Spectroscopy,” Neurology, vol. 43, 2689-95 (1993).

Maker, H. et al., “Regional Changes in Cerebellar Creatine Phosphate Metabolism During Late Maturation,” Exp. Neurol., vol. 38, 295-300 (1973).

Manos, P. et al., “Creatine Kinase Activity in Postnatal Rat Brain Development and in Cultured Neurons, Astrocytes, and Oligodendrocytes,” J. Neurochem., vol. 56, 2101-7 (1991).

Molloy, G. et al., “Rat Brain Creatine Kinase Messenger RNA Levels are High in Primary Cultures of Brain Astrocytes and Oligodencrocytes and Low in Neurons,” J. Neurochem., vol. 59, 1925-32 (1992).

Newman, E., “Regulation of Potassium Levels by Glial Cells in the Retina,” Trends Neurosciencl, vol. 8, 156-9 (1985).

Oblinger, M. et al., “Cytotypic Differences in the Protein Composition of the Axonally Transported Cytoskeleton in Mammalian Neurons,” J. Neurol., vol. 7, No. 2, 453-62 (1987).

Orlovskaia, D. D. et al., “Neuromorphology and Neurochemistry of Senile Dementias in the Light of Studies on Glial Response,” Vestn Ross Akad Med Nauk., vol. 8, 34-9 (1992)—abstract only.

Reichenbach, A., “Glial K+ Permeability and CNS K+ Clearance by Diffusion and Spatial Buffering,” Acad. Sci. New York, 272-86 (1991). Chemical Society, vol. 93, 5542-51 (1971).

Schiffmann, R. et al., “Childhood Ataxia with Diffuse Central Nervous System Hypomyelination,” Ann. Neurol., vol. 35, 331-40 (1994).

Schultz, J. et al., “Blockade of Neuronal Nitric Oxide Synthase Protects Against Excitotoxicity in vivo,” J. Neurosci., vol. 15, No. 12, 8419-29 (1995).

Schultz, J. et al., “Inhibition of Neuronal Nitric Oxide Synthase by 7-Nitroindazole Protects Against MPTP-Induced Neurotoxicity in Mice,” J. Neurochem., vol. 64, 936-9 (1995).

Stadhouders, A., et al., “Mitochondrial Creatine Kinase: A Major Constituent of Pathological Inclusions Seen in Mitochondrial Myopathies,” PNAS, vol. 91, No. 11, 5089-93 (1994).

Wang, T., “Synthesis and Properties of N-Acetimidoyl Derivatives of Glycine and Sarcosine,” JOC, vol. 39, No. 24, 3591-4 (1974).

Beal, M. F., et al, “Do defects in mitochondrial energy metabolism underlie the pathology of neurodegenerative diseases?” Trends Neurosci. 16: 125-131(1993).

Folbergrova, J., et al., “Focal and perifocal changes in tissue energy state during middle cerebral artery occlusion in normo- and hyperglycemic rats,” J. Cereb. Blood Flow Metab. 12: 25-33 (1992).

Ginsberg, M. D. and Busto, R., “Rodent models of cerebrasl ischemia (Progress Review),” Stroke 20:1627-1640 (1989).

Mosinger, J. L. and Olney, J. W., “Photothrombosis-induced ischemic neuronal degeneration in the rat retina,” Exp. Neurol. 105: 110-113, (1989).

J. W., “Excitatory amino acids and neuropsychiatric disorders,” Biol. Psychiatry 26:505-525 (1989).

Olmey. J. W., “NMDA antagonist neurotoxicity: Mechanism and prevention,” Science 254: 1515-1518 (1991).

Siesjo, B. K., Brain Energy Metabolism (John Wiley & Sons, New York, 1978).

Simon, R. P., et al., “Blockade of N-methyl-D-aspartate receptors may protect against ischemic damage in the brain,” Science 226: 850-852 (1984).

Voll, C. L. and Auer, R. N. “Insulin attenuates ischemic brain damage independent of its hypoglycemic effect,” J. Cereb. Blood Flow and Metabolism 11: 1006-1014 (1991).

### SUMMARY OF INVENTION

The present invention pertains to methods of treating diseases or events of the nervous systems in an individual afflicted with such a disease or event by administering to the afflicted individual an amount of a salt of methyl pyruvic acid (such as potassium methyl pyruvate) sufficient to protect against neuronal degeneration thereby, preventing, reducing or ameliorating the symptoms. Typical dosages of a methyl pyruvate salt and/or methyl pyruvic acid will depend on factors such as size, age, health, the disease/event and duration of the disease/event. This treatment is effective when administered on a chronic or acute basis.

A preferred mode of use involves co-administration of methyl pyruvate compounds along with one or more agents that promote energy.

A preferred mode of use involves co-administration of methyl pyruvate compounds along with one or more agents that promote proper mitochondria function while decreasing oxidative stress.

The present invention further pertains to methods of use of methyl pyruvate compounds in combination with vitamins, coenzymes, mineral substances, amino acids, antioxidants, herbs, and creatine compounds, or pharmaceutical drugs which act on the cell for enhancing function and viability.

Compounds effective for this purpose include the present invention, which also provides compositions containing methyl pyruvate compounds in combination with a pharmaceutically acceptable carrier, and effective amounts of other agents, which act on the nervous system, to prophylactically and/or therapeutically treat a subject with a disease of the nervous system.

Some of the diseases susceptible to treatment with methyl pyruvate compounds according to the present invention include, but are not limited to Alzheimer disease, Parkinson's disease, Huntington's disease, motor neuron disease, diabetic and toxic neuropathies, traumatic nerve injury, multiple sclerosis, acute disseminated encephalomyelitis, acute necrotizing hemorrhagic leukoencephalitis, diseases of dysmyelination, mitochondrial diseases, fungal and bacterial infections, migrainous disorders, stroke, aging, dementia, and mental disorders such as depression and schizophrenia. Any diseased caused by an impairment in intracellular energy metabolism, especially if the impairment were in the glycolytic pathway, methyl pyruvate could be administered orally on a chronic basis to maintain energy in CNS neurons at a level that will protect the neuron from degenerating.

The present invention further pertains to methods of use of methyl pyruvate compounds in treatment to protect against neuronal degeneration due to ischemia (inadequate blood flow, which can be caused by stroke, cardiac arrest, or other events) or due to hypoxia, hypoglycemia, or cellular disorders which interfere with the energy metabolism of neurons can be effective even when administered after the onset of an event that triggers acute neurodegeneration. Use of methyl pyruvate can be effective when administered orally or infused on an acute basis. Typical dosages of methyl pyruvate compounds, will depend on factors such as the size and condition of the patient and the amount of time that has elapsed since the onset of the ischemic event.

### DETAILED DESCRIPTION

This invention entails a use of methyl pyruvate to protect against neuronal degeneration. Methyl pruvate is the ionized form of methyl pyruvic acid (CH3C(O)CO2CH3). At physiologic pH, the hydrogen proton dissociates from the carboxylic acid group, thereby generating the methyl pyruvate anion. When used as a pharmaceutical or dietary supplement, this anion can be formulated as a salt, using a monovalent or divalent cation such as sodium, potassium, magnesium, or calcium.

Pancreatic beta-cell as a model: The energy requirements of most cells supplied with glucose are fulfilled by glycolytic and oxidative metabolism, yielding ATP. When cytosolic and mitochondrial contents in ATP, ADP and AMP were measured in islets incubated for 45 min at increasing concentrations of D-glucose and then exposed for 20 s to digitonin. The latter treatment failed to affect the total islet ATP/ADP ratio and adenylate charge. D-Glucose caused a much greater increase in cytosolic than mitochondrial ATP/ADP ratio. In the cytosol, a sigmoidal pattern characterized the changes in ATP/ADP ratio at increasing concentrations of D-glucose. These findings are compatible with the view that cytosolic ATP participates in the coupling of metabolic to ionic events in the process of nutrient-induced insulin release.

To gain insight into the regulation of pancreatic beta-cell mitochondrial metabolism, the direct effects on respiration of different mitochondrial substrates, variations in the ATP/ADP ratio and free Ca2+ were examined using isolated mitochondria and permeabilized clonal pancreatic beta-cells (HIT). Respiration from pyruvate was highand not influenced by Ca2+ in State 3 or under various redox states and fixed values of the ATP/ADP ratio; nevertheless, high Ca2+ elevated pyridine nucleotide fluorescence, indicating activation of pyruvate dehydrogenase by Ca2+. Furthermore, in the presence of pyruvate, elevated Ca2+ stimulated CO2 production from pyruvate, increased citrate production and efflux from the mitochondria and inhibited CO2 production from palmitate. The latter observation suggests that beta-cell fatty acid oxidation is not regulated exclusively by malonyl-CoA but also by the mitochondrial redox state. alpha-Glycerophosphate (alpha-GP) oxidation was Ca(2+)-dependent with a half-maximal rate observed at around 300 nM Ca2+. It was recently demonstrated that increases in respiration precede increases in Ca2+ in glucose-stimulated clonal pancreatic beta-cells (HIT), indicating that Ca2+ is not responsible for the initial stimulation of respiration. It is suggested that respiration is stimulated by increased substrate (alpha-GP and pyruvate) supply together with oscillatory increases in ADP.

The rise in Ca2+, which in itself may not significantly increase net respiration, could have the important functions of: (1) activating the alpha-GP shuttle, to maintain an oxidized cytosol and high glycolytic flux; (2) activating pyruvate dehydrogenase, and indirectly pyruvate carboxylase, to sustain production of citrate and hence the putative signal coupling factors, malonyl-CoA and acyl-CoA; (3) increasing mitochondrial redox state to implement the switch from fatty acid to pyruvate oxidation.

Glucose-stimulated increases in mitochondrial metabolism are generally thought to be important for the activation of insulin secretion. Pyruvate dehydrogenase (PDH) is a key regulatory enzyme, believed to govern the rate of pyruvate entry into the citrate cycle. It has been shown that elevated glucose concentrations (16 or 30 vs 3 mM) cause an increase in PDH activity in both isolated rat islets, and in a clonal beta-cell line (MIN6). However, increases in PDH activity elicited with either dichloroacetate, or by adenoviral expression of the catalytic subunit of pyruvate dehydrogenase phosphatase, were without effect on glucose-induced increases in mitochondrial pyridine nucleotide levels, or cytosolic ATP concentration, in MIN6 cells, and insulin secretion from isolated rat islets. Similarly, the above parameters were unaffected by blockade of the glucose-induced increase in PDH activity by adenovirus-mediated over-expression of PDH kinase (PDK). Thus, activation of the PDH complex plays an unexpectedly minor role in stimulating glucose metabolism and in triggering insulin release.

In pancreatic beta-cells, a rise in cytosolic ATP is also a critical signaling event, coupling closure of ATP-sensitive K+ channels (KATP) to insulin secretion via depolarization-driven increases in intracellular Ca2+. Glycolytic but not Krebs cycle metabolism of glucose is critically involved in this signaling process.

While inhibitors of glycolysis suppressed glucose-stimulated insulin secretion, blockers of pyruvate transport or Krebs cycle enzymes were without effect. While pyruvate was metabolized in islets to the same extent as glucose, it produced no stimulation of insulin secretion and did not block KATP.

In pancreatic beta-cells, methyl pyruvate is a potent secretagogue and is widely used to study stimulus-secretion coupling. MP stimulated insulin secretion in the absence of glucose, with maximal effect at 5 mM. MP depolarized the beta-cell in a concentration-dependent manner (5-20 mM). Pyruvate failed to initiate insulin release (5-20 mM) or to depolarize the membrane potential. ATP production in isolated beta-cell mitochondria was detected as accumulation of ATP in the medium during incubation in the presence of malate or glutamate in combination with pyruvate or MP. ATP production by MP and glutamate was higher than that induced by pyruvate/glutamate. Pyruvate (5 mM) or MP (5 mM) had no effect on the ATP/ADP ratio in whole islets, whereas glucose (20 mM) significantly increased the whole islet ATP/ADP ratio.

In contrast with pyruvate, which barely stimulates insulin secretion, methyl pyruvate was suggested to act as an effective mitochondrial substrate. Methyl pyruvate elicited electrical activity in the presence of 0.5 mM glucose, in contrast with pyruvate. Accordingly, methyl pyruvate increased the cytosolic free Ca(2+) concentration after an initial decrease, similar to glucose. However, in contrast with glucose, methyl pyruvate even slightly decreased NAD(P)H autofluorescence and did not influence ATP production or the ATP/ADP ratio. Therefore, MP-induced beta-cell membrane depolarization or insulin release does not relate directly to mitochondrial ATP production.

The finding that methyl pyruvate directly inhibited a cation current across the inner membrane of Jurkat T-lymphocyte mitochondria suggests that this metabolite may increase ATP production in beta-cells by activating the respiratory chains without providing reduction equivalents. This mechanism may account for a slight and transient increase in ATP production. Furthermore methyl pyruvate inhibited the K(ATP) current measured in the standard whole-cell configuration. Accordingly, single-channel currents in inside-out patches were blocked by methyl pyruvate. Therefore, the inhibition of K(ATP) channels, and not activation of metabolism, mediates the induction of electrical activity in pancreatic beta-cells by methyl pyruvate.

As a membrane-permeant analog, methyl pyruvate, produced a block of KATP, a sustained rise in [Ca2+]i, and an increase in insulin secretion 6-fold the magnitude of that induced by glucose. This indicates that ATP derived from mitochondrial pyruvate metabolism does not substantially contribute to the regulation of KATP responses to a glucose challenge. Supporting the notion of sub-compartmentation of ATP within the beta-cell. Supra-normal stimulation of the Krebs cycle by methyl pyruvate can, however, overwhelm intracellular partitioning of ATP and thereby drive insulin secretion.

The metabolism of methyl pyruvate was compared to that of pyruvate in isolated rat pancreatic islets. Methyl pyruvate was found to be more efficient than pyruvate in supporting the intramitochondrial conversion of pyruvate metabolites to amino acids, inhibiting D-[5-3H]glucose utilization, maintaining a high ratio between D-[3,4-14C] glucose or D-[6-14C]glucose oxidation and D-[5-3H]glucose utilization, inhibiting the intramitochondrial conversion of glucose-derived 2-keto acids to their corresponding amino acids, and augmenting 14CO2 output from islets prelabeled with L-[U-14C] glutamine. Methyl pyruvate also apparently caused a more marked mitochondrial alkalinization than pyruvate, as judged from comparisons of pH measurements based on the use of either a fluorescein probe or 14C-labeled 5,5-dimethyl-oxazolidine-2,4-dione. Inversely, pyruvate was more efficient than methyl pyruvate in increasing lactate output and generating L-alanine. These converging findings indicate that, by comparison with exogenous pyruvate, its methyl ester is preferentially metabolized in the mitochondrial, rather than cytosolic, domain of islet cells. It is proposed that both the positive and the negative components of methyl pyruvate insulinotropic action are linked to changes in the net generation of reducing equivalents, ATP and H+.

Methyl pyruvate was found to exert a dual effect on insulin release from isolated rat pancreatic islets. A positive insulinotropic action prevailed at low concentrations of D-glucose, in the 2.8 to 8.3 mM range, and at concentrations of the ester not exceeding 10.0 mM. It displayed features typical of a process of nutrient-stimulated insulin release, such as decreased K+ conductance, enhanced Ca2+ influx, and stimulation of proinsulin biosynthesis. A negative insulinotropic action of methyl pyruvate was also observed, however, at a high concentration of D-glucose (16.7 mM) and/or at a high concentration of the methyl ester (20.0 mM). It was apparently not attributable to any adverse effect of methyl pyruvate on ATP generation, but might be due to hyperpolarization of the plasma membrane. The ionic determinant(s) of the latter change was not identified. The dual effect of methyl pyruvate probably accounts for an unusual time course of the secretory response, including a dramatic and paradoxical stimulation of insulin release upon removal of the ester.

Pancreatic beta-cell metabolism was followed during glucose and pyruvate stimulation of pancreatic islets using quantitative two-photon NAD(P)H imaging. The observed redox changes, spatially separated between the cytoplasm and mitochondria, were compared with whole islet insulin secretion. As expected, both NAD(P)H and insulin secretion showed sustained increases in response to glucose stimulation. In contrast, pyruvate caused a much lower NAD(P)H response and did not generate insulin secretion. Low pyruvate concentrations decreased cytoplasmic NAD(P)H without affecting mitochondrial NAD(P)H, whereas higher concentrations increased cytoplasmic and mitochondrial levels. However, the pyruvate-stimulated mitochondrial increase was transient and equilibrated to near-base-line levels. Inhibitors of the mitochondrial pyruvate-transporter and malate-aspartate shuttle were utilized to resolve the glucose- and pyruvate-stimulated NAD(P)H response mechanisms.

These data showed that glucose-stimulated mitochondrial NAD(P)H and insulin secretion are independent of pyruvate transport but dependent on NAD(P)H shuttling. In contrast, the pyruvate-stimulated cytoplasmic NAD(P)H response was enhanced by both inhibitors. Surprisingly the malate-aspartate shuttle inhibitor enabled pyruvate-stimulated insulin secretion. These data support a model in which glycolysis plays a dominant role in glucose-stimulated insulin secretion. Based on these data, it was proposed as a mechanism for glucose-stimulated insulin secretion that includes allosteric inhibition of tricarboxylic acid cycle enzymes and pH dependence of mitochondrial pyruvate transport.

Pyridine dinucleotides (NAD and NADP) are ubiquitous cofactors involved in hundreds of redox reactions essential for the energy transduction and metabolism in all living cells. In addition, NAD also serves as a substrate for ADP-ribosylation of a number of nuclear proteins, for silent information regulator 2 (Sir2)-like histone deacetylase that is involved in gene silencing regulation, and for cyclic ADP ribose (cADPR)-dependent Ca(2+) signaling. Pyridine nucleotide adenylyltransferase (PNAT) is an indispensable central enzyme in the NAD biosynthesis pathways catalyzing the condensation of pyridine mononucleotide (NMN or NaMN) with the AMP moiety of ATP to form NAD (or NaAD).

1. In isolated pancreatic islets, pyruvate causes a shift to the left of the sigmoidal curve relating the rate of insulin release to the ambient glucose concentration. The magnitude of this effect is related to the concentration of pyruvate (5-90 mM) and, at a 30 mM concentration, is equivalent to that evoked by 2 mM-glucose.

2. In the presence of glucose 8 mM), the secretory response to pyruvate is an immediate process, displaying a biphasic pattern.

3. The insulinotropic action of pyruvate coincides with an inhibition of 45 Ca efflux and a stimulation of 45 Ca net uptake. The relationship between 45 Ca uptake and insulin release displays its usual pattern in the presence of pyruvate.

4. Exogenous pyruvate rapidly accumulates in the islets in amounts close to those derived from the metabolism of glucose. The oxidation of [2-14C] pyruvate represents 64% of the rate of [1-14C] pyruvate decarboxylation and, at a 30 mM concentration, is comparable with that of 8 mM-[U-14C]glucose.

5. When corrected for the conversion of pyruvate into lactate, the oxidation of 30 mM-pyruvate corresponds to a net generation of about 314 pmol of reducing equivalents/120 min per islet.

6. Pyruvate does not affect the rate of glycolysis, but inhibits the oxidation of glucose. Glucose does not affect pyruvate oxidation.

7. Pyruvate (30 mM) does not affect the concentration of ATP, ADP and AMP in the islet cells.

8. Pyruvate (30 mM) increases the concentration of reduced nicotinamide nucleotides in the presence but not in the absence of glucose. A close correlation is seen between the concentration of reduced nicotinamide nucleotides and the net uptake of 45 Ca.

9. Pyruvate, like glucose, modestly stimulates lipogenesis.

10. Pyruvate, in contrast with glucose, markedly inhibits the oxidation of endogenous nutrients. The latter effect accounts for the apparent discrepancy between the rate of pyruvate oxidation and the magnitude of its insulinotropic action.

11. It is concluded that the effect of pyruvate to stimulate insulin release depends on its ability to increase the concentration of reduced nicotinamide nucleotides in the islet cells.

Glucose-stimulated insulin secretion is a multi-step process dependent on cell metabolic flux. Previous studies on intact pancreatic islets used two-photon NAD(P)H imaging as a quantitative measure of the combined redox signal from NADH and NADPH (referred to as NAD(P)H). These studies showed that pyruvate, a non-secretagogue, enters -cells and causes a transient rise in NAD(P)H. To further characterize the metabolic fate of pyruvate, a one-photon flavoprotein microscopy has been developed as a simultaneous assay of lipoamide dehydrogenase (LipDH) autofluorescence. This flavoprotein is in direct equilibrium with mitochondrial NADH.

Using this method, the glucose-dose response is consistent with an increase in both NADH and NADPH. In contrast, the transient rise in NAD(P)H observed with pyruvate stimulation is not accompanied by a significant change in LipDH, which indicates that pyruvate raises cellular NADPH without raising NADH. In comparison, methyl pyruvate stimulated a robust NADH and NADPH response. These data provide new evidence that exogenous pyruvate does not induce a significant rise in mitochondrial NADH. This inability likely results in its failure to produce the ATP necessary for stimulated secretion of insulin. Overall, these data are consistent with either restricted PDH dependent metabolism or a buffering of the NADH response by other metabolic mechanisms.

Glucose metabolism in glycolysis and in mitochondria is pivotal to glucose-induced insulin secretion from pancreatic beta cells. One or more factors derived from glycolysis other than pyruvate appear to be required for the generation of mitochondrial signals that lead to insulin secretion. The electrons of the glycolysis-derived reduced form of nicotinamide adenine dinucleotide (NADH) are transferred to mitochondria through the NADH shuttle system. By abolishing the NADH shuttle function, glucose-induced increases in NADH autofluorescence, mitochondrial membrane potential, and adenosine triphosphate content were reduced and glucose-induced insulin secretion was abrogated. The NADH shuttle evidently couples glycolysis with activation of mitochondrial energy metabolism to trigger insulin secretion.

To determine the role of the NADH shuttle system composed of the glycerol phosphate shuttle and malate-aspartate shuttle in glucose-induced insulin secretion from pancreatic beta cells, mice which lack mitochondrial glycerol-3 phosphate dehydrogenase mGPDH), a rate-limiting enzyme of the glycerol phosphate shuttle were used. When both shuttles were halted in mGPDH-deficient islets treated with aminooxyacetate, an inhibitor of the malate-aspartate shuttle, glucose-induced insulin secretion was almost completely abrogated. Under these conditions, although the flux of glycolysis and supply of glucose-derived pyruvate into mitochondria were unaffected, glucose-induced increases in NAD(P)H autofluorescence, mitochondrial membrane potential, Ca2+ entry into mitochondria, and ATP content were severely attenuated.

This study provides the first direct evidence that the NADH shuttle system is essential for coupling glycolysis with the activation of mitochondrial energy metabolism to trigger glucose-induced insulin secretion and thus revises the classical model for the metabolic signals of glucose-induced insulin secretion.

Incubation of porcine carotid arteries with 0.4 mmol amino-oxyacetic acid an inhibitor of glutamate-oxaloacetate transaminase and, hence the malate-aspartate shuttle, inhibited O2 consumption by 21%, decreased the content of phosphocreatine and inhibited activity of the tricarboxylic acid cycle. The rate of glycolysis and lactate production was increased but glucose oxidation was inhibited. These effects of amino-oxyacetic acid were accompanied by evidence of inhibition of the malate-aspartate shuttle and elevation in the cytoplasmic redox potential and NADH/NAD ratio as indicated by elevation of the concentration ratios of the lactate/pyruvate and glycerol-3-phosphate/dihydroxyacetone phosphate metabolite redox couples. Addition of the fatty acid octanoate normalized the adverse energetic effects of malate-aspartate shuttle inhibition. It is concluded that the malate-aspartate shuttle is a primary mode of clearance of NADH reducing equivalents from the cytoplasm in vascular smooth muscle. Glucose oxidation and lactate production are influenced by the activity of the shuttle. The results support the hypothesis that an increased cytoplasmic NADH redox potential impairs mitochondrial energy metabolism.

Beta-Methyleneaspartate, a specific inhibitor of aspartate aminotransferase (EC 2.6.1.1.), was used to investigate the role of the malate-aspartate shuttle in rat brain synaptosomes. Incubation of rat brain cytosol, “free” mitochondria, synaptosol, and synaptic mitochondria, with 2 mM beta-methyleneaspartate resulted in inhibition of aspartate aminotransferase by 69%, 67%, 49%, and 76%, respectively. The reconstituted malate-aspartate shuttle of “free” brain mitochondria was inhibited by a similar degree (53%). As a consequence of the inhibition of the aspartate aminotransferase, and hence the malate-aspartate shuttle, the following changes were observed in synaptosomes: decreased glucose oxidation via the pyruvate dehydrogenase reaction and the tricarboxylic acid cycle; decreased acetylcholine synthesis; and an increase in the cytosolic redox state, as measured by the lactate/pyruvate ratio. The main reason for these changes can be attributed to decreased carbon flow through the tricarboxylic acid cycle (i.e., decreased formation of oxaloacetate), rather than as a direct consequence of changes in the NAD+/NADH ratio. Malate/glutamate oxidation in “free” mitochondria was also decreased in the presence of 2 mM beta-methyleneaspartate. This is probably a result of decreased glutamate transport into mitochondria as a result of low levels of aspartate, which are needed for the exchange with glutamate by the energy-dependent glutamate-aspartate translocator.

Aminooxyacetate, an inhibitor of pyridoxal-dependent enzymes, is routinely used to inhibit gamma-aminobutyrate metabolism. The bioenergetic effects of the inhibitor on guinea-pig cerebral cortical synaptosomes are investigated. It prevents the reoxidation of cytosolic NADH by the mitochondria by inhibiting the malate-aspartate shuttle, causing a 26 mV negative shift in the cytosolic NAD+/NADH redox potential, an increase in the lactate/pyruvate ratio and an inhibition of the ability of the mitochondria to utilize glycolytic pyruvate. The 3-hydroxybutyrate/acetoacetate ratio decreased significantly, indicating oxidation of the mitochondrial NAD+/NADH couple. The results are consistent with a predominant role of the malate-aspartate shuttle in the reoxidation of cytosolic NADH in isolated nerve terminals. Aminooxyacetate limits respiratory capacity and lowers mitochondrial membrane potential and synaptosomal ATP/ADP ratios to an extent similar to glucose deprivation.

Variations in the cytoplasmic redox potential (Eh) and NADH/NAD ratio as determined by the ratio of reduced to oxidized intracellular metabolite redox couples may affect mitochondrial energetics and alter the excitability and contractile reactivity of vascular smooth muscle. To test these hypotheses, the cytoplasmic redox state was experimentally manipulated by incubating porcine carotid artery strips in various substrates. The redox potentials of the metabolite couples [lactate]/[pyruvate]i and [glycerol 3-phosphate]/[dihydroxyacetone phosphate]i varied linearly (r=0.945), indicating equilibrium between the two cytoplasmic redox systems and with cytoplasmic NADH/NAD. Incubation in physiological salt solution (PSS) containing 10 mm pyruvate ([lact]/[pyr]=0.6) increased O2 consumption approximately 45% and produced anaplerosis of the tricarboxylic acid (TCA cycle), whereas incubation with 10 mm lactate-PSS ([lact]/[pyr]i=47) was without effect. A hyperpolarizing dose of external KCl (10 mM) produced a decrease in resting tone of muscles incubated in either glucose-PSS (−0.8+/−0.8 g) or pyruvate-PSS (−2.1+/−0.8 g), but increased contraction in lactate-PSS (1.5+/−0.7 g) (n=12-18, P<0.05). The rate and magnitude of contraction with 80 mm KCl (depolarizing) was decreased in lactate-PSS (P=0.001). Slopes of KCl concentration-response curves indicated pyruvate >glucose >lactate (P<0.0001); EC50 in lactate (29.1+/−1.0 mM) was less than that in either glucose (32.1±0.9 mm) or pyruvate (32.2+/−1.0 mM), P<0.03. The results are consistent with an effect of the cytoplasmic redox potential to influence the excitability of the smooth muscle and to affect mitochondrial energetics.

The cytoplasmic NADH/NAD redox potential affects energy metabolism and contractile reactivity of vascular smooth muscle. NADH/NAD redox state in the cytosol is predominately determined by glycolysis, which in smooth muscle is separated into two functionally independent cytoplasmic compartments, one of which fuels the activity of Na(+)-K(+)-ATPase. The effect was examined of varying the glycolytic compartments on cystosolic NADH/NAD redox state. Inhibition of Na(+)-K(+)-ATPase by 10 microM ouabain resulted in decreased glycolysis and lactate production. Despite this, intracellular concentrations of the glycolytic metabolite redox couples of lactate/pyruvate and glycerol-3-phosphate/dihydroxyacetone phosphate (thus NADH/NAD) and the cytoplasmic redox state were unchanged. The constant concentration of the metabolite redox couples and redox potential was attributed to: 1) decreased efflux of lactate and pyruvate due to decreased activity of monocarboxylate B—H(+) transporter secondary to decreased availability of H(+) for cotransport and 2) increased uptake of lactate (and perhaps pyruvate) from the extracellular space, probably mediated by the monocarboxylate-H(+) transporter, which was specifically linked to reduced activity of Na(+)-K(+)-ATPase. It was concluded that redox potentials of the two glycolytic compartments of the cytosol maintain equilibrium and that the cytoplasmic NADH/NAD redox potential remains constant in the steady state despite varying glycolytic flux in the cytosolic compartment for Na(+)-K(+)-ATPase

Poly(ADP-ribose) polymerases (PARPs) are defined as cell signaling enzymes that catalyze the transfer of ADP-ribose units from NAD(+)to a number of acceptor proteins. PARP-1, the best characterized member of the PARP family, that presently includes six members, is an abundant nuclear enzyme implicated in cellular responses to DNA injury provoked by genotoxic stress (oxygen radicals, ionizing radiations and monofunctional alkylating agents). Due to its involvement either in DNA repair or in cell death, PARP-1 is regarded as a double-edged regulator of cellular functions. In fact, when the DNA damage is moderate, PARP-1 participates in the DNA repair process. Conversely, in the case of massive DNA injury, elevated PARP-1 activation leads to rapid NAD(+)/ATP consumption and cell death by necrosis.

Peroxynitrite and hydroxyl radicals are potent initiators of DNA single-strand breakage, which is an obligatory stimulus for the activation of the nuclear enzyme poly(ADP ribose) polymerase (PARP). In response to high glucose incubation medium in vitro, or diabetes and hyperglycemia in vivo, reactive nitrogen and oxygen species generation occurs. These reactive species trigger DNA single-strand breakage, which induces rapid activation of PARP. PARP in turn depletes the intracellular concentration of its substrate, NAD+, slowing the rate of glycolysis, electron transport, and ATP formation. This process results in acute endothelial dysfunction in diabetic blood vessels.

PARP-1 functions as a DNA damage sensor and signalling molecule. Upon binding to DNA breaks, activated PARP cleaves NAD(+)nicotinamide and ADP-ribose and polymerizes the latter onto nuclear acceptor proteins including histones, transcription factors and PARP itself. Poly(ADP-ribosylation) contributes to DNA repair and to the maintenance of genomic stability. On the other hand, oxidative stress-induced overactivation of PARP consumes NAD(+) and consequently ATP, culminating in cell dysfunction or necrosis. This cellular suicide mechanism has been implicated in the pathomechanism of stroke, myocardial ischemia, diabetes, diabetes-associated cardiovascular dysfunction, shock, traumatic central nervous system injury, arthritis, colitis, allergic encephalomyelitis, and various other forms of inflammation.

Methyl pyruvate has been described with reference to a particular embodiment. For one skilled in the art, other modifications and enhancements can be made without departing from the spirit and scope of the aforementioned claims.

Whilst endeavoring in the foregoing Specification to draw attention to those features of the invention believed to be of particular importance it should be understood that the Applicant claims protection in respect of any patentable feature hereinbefore referred to whether or not particular emphasis has been placed thereon.

What is claimed is:

1. We claim a method of increasing neuronal energy production with the use of methyl pyruvate in a human.

2. We claim a method of increasing neuronal energy production with the use of methyl pyruvic acid in a human.

3. We claim a method of increasing methyl pyruvate levels and said effects in a human.

4. We claim a method of increasing methyl pyruvic acid levels and said effects in a human.

5. We claim the method of claim 2 wherein a therapeutic and effective amount of methyl pyruvic acid is infused or orally administered to the human.

6. We claim the method of claim 1 wherein a therapeutic and effective amount of the salt of methyl pyruvate is infused or orally administered to the human.

7. We claim the method of claim 6 wherein the salt of methyl pyruvate is a monovalent cation (such as sodium or potassium methyl pyruvate).

8. We claim the method of claim 6 wherein the salt of methyl pyruvate is a divalent cation (such as calcium or magnesium methyl pyruvate).

9. We claim the method of claim 6 wherein analogs of these compounds can act as substrates or substrate analogs for methyl pyruvate.

10. We claim the method of claim 6 wherein the salt of methyl pyruvate and composition of a pharmacologically acceptable excipient and/or diluent therefore.

11. We claim the method of claim 9 wherein the salt of methyl pyruvate and composition which further may comprise vitamins, coenzymes, mineral substances, amino acids, herbs and antioxidants or pharmaceutical drugs.

12. We claim the method of claim 10, infused or orally administrable, in the form of a dietary supplement, energizer or pharmaceutical drug.

13. We claim the method of claim 11, infused or orally administrable, in the form of a dietary supplement, energizer or pharmaceutical drug.

14. We claim the method of claim 12, in the form of lozenges, tablets, pills, capsules, powders, granulates, sachets, syrups or vials.

15. We claim the method of claim 13, in the form of lozenges, tablets, pills, capsules, powders, granulates, sachets, syrups or vials.

16. We claim the method of claim 14, in unit dosage form, comprising from about 100 mg to about 28 grams, preferably between about 0.5 grams-5 grams.

17. We claim the method of claim 15, in unit dosage form, comprising from about 100 mg to about 28 grams, preferably between about 0.5 grams-5 grams.

18. We claim the method of claim 17, for treating a subject afflicted with amyotropic lateral sclerosis, comprising administering to the subject an amount of methyl pyruvate salt, such that the subject is treated for amyotropic lateral sclerosis.

19. We claim the method of claim 17, for treating a subject afflicted with Parkinson's disease, comprising administering to the subject an amount methyl pyruvate salt, such that the subject is treated for Parkinson's disease.

20. We claim the method of claim 17, for treating a subject afflicted with Huntington's disease, comprising administering to the subject an amount of methyl pyruvate salt, such that the subject is treated for Huntington's disease.

21. We claim the method of claim 17, for treating a subject afflicted with Alzheimer's disease, comprising administering to the subject an amount of methyl pyruvate salt, such that the subject is treated for Alzheimer's disease.

22. We claim the method of claim 17, for treating a subject afflicted with multiple sclerosis, comprising administering to the subject an amount of methyl pyruvate salt, such that the subject is treated for multiple sclerosis.

23. We claim the method of claim 5 wherein analogs can act as substrates or substrate analogs for methyl pyruvic acid.

24. We claim the method of claim 5 wherein methyl pyruvic acid and composition of a pharmacologically acceptable excipient and/or diluent therefore.

25. We claim the method of claim 23 wherein methyl pyruvic acid and composition which further may comprise vitamins, coenzymes, mineral substances, amino acids, herbs and antioxidants or pharmaceutical drugs.

26. We claim the method of claim 24, infused or orally administrable, in the form of a dietary supplement, energizer or pharmaceutical drug.

27. We claim the method of claim 25, infused or orally administrable, in the form of a dietary supplement, energizer or pharmaceutical drug.

28. We claim the method of claim 26, in the form of lozenges, tablets, pills, capsules, powders, granulates, sachets, syrups or vials.

29. We claim the method of claim 27, in the form of lozenges, tablets, pills, capsules, powders, granulates, sachets, syrups or vials.

30. We claim the method of claim 28, in unit dosage form, comprising from about 100 mg to about 28 grams, preferably between about 0.5 grams-5 grams.

31. We claim the method of claim 29, in unit dosage form, comprising from about 100 mg to about 28 grams, preferably between about 0.5 grams-5 grams.

32. We claim the method of claim 31, for treating a subject afflicted with amyotropic lateral sclerosis, comprising administering to the subject an amount of methyl pyruvic acid, such that the subject is treated for amyotropic lateral sclerosis.

33. We claim the method of claim 31, for treating a subject afflicted with Parkinson's disease, comprising administering to the subject an amount of methyl pyruvic acid, such that the subject is treated for Parkinson's disease.

34. We claim the method of claim 31, for treating a subject afflicted with Huntington's disease, comprising administering to the subject an amount of methyl pyruvic acid, such that the subject is treated for Huntington's disease.

35. We claim the method of claim 31, for treating a subject afflicted with Alzheimer's disease, comprising administering to the subject an amount of methyl pyruvic acid, such that the subject is treated for Alzheimer's disease.

36. We claim the method of claim 31, for treating a subject afflicted with multiple sclerosis, comprising administering to the subject an amount of methyl pyruvic acid, such that the subject is treated for multiple sclerosis.

37. We claim the method of claim 17, for protecting a human central nervous system against neuronal degeneration caused by a defect in at least one intracellular energy metabolic enzyme, comprising the step of administering to a human at risk of such neuronal degeneration a therapeutically effective quantity of said substance to neurons to promote oxidative metabolism.

38. We claim the method of claim 31, for protecting a human central nervous system against neuronal degeneration caused by a defect in at least one intracellular energy metabolic enzyme, comprising the step of administering to a human at risk of such neuronal degeneration a therapeutically effective quantity of said substance to neurons to promote oxidative metabolism.

39. We claim the method of claim 17, which further comprises Creatine compounds, which can be used in the present method include (1) creatine, creatine phosphate and analogs of these compounds which can act as substrates or substrate analogs for creatine kinase; (2) bisubstrate inhibitors of creatine kinase comprising covalently linked structural analogs of adenosine triphosphate (ATP) and creatine; (3) creatine analogs which can act as reversible or irreversible inhibitors of creatine kinase; and (4) N-phosphorocreatine analogs bearing non-transferable moieties which mimic the N-phosphoryl group.

40. We claim the method of claim 31, which further comprises Creatine compounds, which can be used in the present method include (1) creatine, creatine phosphate and analogs of these compounds which can act as substrates or substrate analogs for creatine kinase; (2) bisubstrate inhibitors of creatine kinase comprising covalently linked structural analogs of adenosine triphosphate (ATP) and creatine; (3) creatine analogs which can act as reversible or irreversible inhibitors of creatine kinase; and (4) N-phosphorocreatine analogs bearing non-transferable moieties which mimic the N-phosphoryl group.

41. We claim the method of claim 17, for protecting a human central nervous system against neuronal degeneration triggered by an ischemic event, comprising the step of injecting, into the bloodstream of a human at risk of ischemic damage, a therapeutically effective quantity.

42. We claim the method of claim 31, for protecting a human central nervous system against neuronal degeneration triggered by an ischemic event, comprising the step of injecting, into the bloodstream of a human at risk of ischemic damage, a therapeutically effective quantity.

43. We claim the method of claim 41 wherein administered to the human in conjunction with insulin.

44. We claim the method of claim 42 wherein administered to the human in conjunction with insulin.
